(19) United States
(12) Patent Application Publication
(10) Pub. No.: US 2018/0071133 A1
(43) Pub. Date: Mar. 15, 2018

(54) A SNORE DISRUPTION SYSTEM

(71) Applicants: Aliasghar HARIRI, Toronto (CA); Sahar HARIRI, Toronto (CA)

(72) Inventors: Aliasghar HARIRI, Toronto (CA); Sahar HARIRI, Toronto (CA)

(21) Appl. No.: 15/558,266

(22) PCT Filed: Jul. 7, 2015
(86) PCT No.: PCT/CA2015/050630

§ 371 Date: Sep. 14, 2017

(51) Int. Cl. A61F 5/56; A61B 5/00; A61B 7/00; A47G 9/10

(52) U.S. Cl. CPC A61F 5/56; A61B 5/4818; A61B 5/7282; A61B 7/003; A47G 9/1027; A61B 5/4836

(57) ABSTRACT

A snore disrupting method and system is presented, comprising an expansion unit, an air pressure supply or an inflator unit, and an electronic circuit. The invention may further comprise a wired or wireless microphone transmitter. The expansion unit may be placed underneath a pillow, inside a pillowcase. The expansion unit is inflated and deflated by the inflator unit and through expanding and increasing in volume and deflating and reducing in volume changes the position of snorer's head, hence disrupting the snoring. The expansion unit may comprise at least one bladder placed within a sleeve structure, where the expansion and volume increase of the one or plurality of bladders expands the sleeve structure as well and extends and transfers the expansion through the length of the sleeve structure, therefore displacing the pillow along the length of the sleeve structure.

### FIELD OF INVENTION

The present invention relates generally to a snore disrupting method and system. More specifically, the present invention relates to a system and method for disrupting and stopping snoring, by changing the position of snorer's head when snoring.

### BACKGROUND OF THE INVENTION

Snoring is the vibration of respiratory structures and the resulting sound. The irregular airflow is caused by a passageway blockage and is mostly due to relaxing throat muscles and in some cases is extended to Obstructive Sleep Apnea (OSA).

Statistically, at least 30% of adults snore and rising to 60% of men and 40% of women aged 60 to 65 years; this suggests an increased susceptibility to snoring as age increases.

Snoring is known to cause sleep deprivation to snorers and those around them, as well as daytime drowsiness, irritability, lack of focus and decreased libido. It has also been suggested that it can cause significant psychological and social damage to sufferers. Multiple studies reveal a positive correlation between loud snoring and increased risk of heart attack by about 34%, and increased risk of stroke by about 67% chance.

Many methods have been invented to prevent snoring. U.S. Pat. No. 7,676,870B2 presets a pillow body with base, mobile seat, gear set, and motor assembly, which shifts horizontally to right and left when the sensors within the pillow body detect snoring sound.

U.S. Pat. No. 4,941,478A presents mechanical pillow body that changes the angular position of the pillow surface about a horizontal centerline, when snoring is detected. Canadian Patent 202740193U presents a pillow body with a voice-controlled vibrator inside that vibrates when detecting snoring sound.

The above-mentioned methods and systems have many disadvantages. They are all contained pillow bodies limiting the pillow options of the user, where the user might have medical or personal preference choosing his/her pillow based on a range of features including size, softness, hardness, rigidity, neck support, air flow, material allergies, etc. In some prior art methods and systems the displacement component is isolated with limited effective area and may not be effective if the head is not placed directly on top of the displacement component and elsewhere along the length of the pillow. Furthermore, as enclosed pillow bodies, the mentioned systems place the mechanical and hydraulic components of the system, such as pumps and motors, within the pillow body, resulting in loud noise in close proximity of the user's head. This noise results in disrupting the sleep of the snorer as well as other individuals in the proximity of the system. Including the battery and electrical components within the pillow body results in exposing the head of the user to an electromagnetic field, which can result in headaches and other symptoms.

Therefore it would be desirable to provide a new and advantageous snore-disrupting system.

### BRIEF SUMMARY OF THE INVENTION

It is an object of the present invention to provide a novel system and method for disrupting snore by changing the position of snorer's head, which obviates or mitigates at least one disadvantage of the prior art.

According to the first aspect of the present invention, there is provided a method of disrupting snore comprising an expansion unit, an air pressure supply or an inflator unit, and an electronic circuit. The invention may further comprise a wired or wireless audio-sensor and transmitter or use of a cellphone or any other device that would allow for audio sensing and analog or digital transmission. The expansion unit may be placed underneath a pillow, inside a pillowcase. The expansion unit is inflated and deflated by the inflator unit and through expanding and increasing in volume and deflating and reducing in volume changes the position of snorer's head, hence disrupting the snoring. The expansion unit may comprise at least one bladder placed within a sleeve structure, where the expansion and volume increase of the one or plurality of bladders expands the sleeve structure as well and extends and transfers the expansion through the length of the sleeve structure, therefore displacing the pillow along the length of the sleeve structure.

Thus, the present invention introduces a novel snore disrupting system, where the expansion unit longitudinally extends and transfers an isolated expansion to allow for effective displacement along the length of a pillow used in conjunction with the system.

The present invention further does not comprise a pillow body and therefore accommodate pillow options to be used with the system. This also makes the devise portable and modular in regards to the pillow bodies to be used.

In the present invention no mechanical or electrical components is places in the pillow and under the head of the user, where the expansion unit is the only component of the system that is places in the pillow or in immediate proximity of the head of the user, drastically reducing the issues of system noise and electromagnetic field exposure.

### BRIEF DESCRIPTION OF THE DRAWINGS

Preferred embodiments of the present invention will now be described, by way of example only, with reference to attached Figures, wherein:

FIG. 1: shows a schematic view illustrating a preferred embodiment of the present snore disrupting system.

FIG. 2: shows a schematic view illustrating an embodiment of the expansion unit of the present snore disrupting system, in a maximum inflation position.

FIG. 3: shows a schematic view illustrating an embodiment of the expansion unit of the present snore disrupting system, in a maximum deflation position.

### DETAILED DESCRIPTION OF THE INVENTION

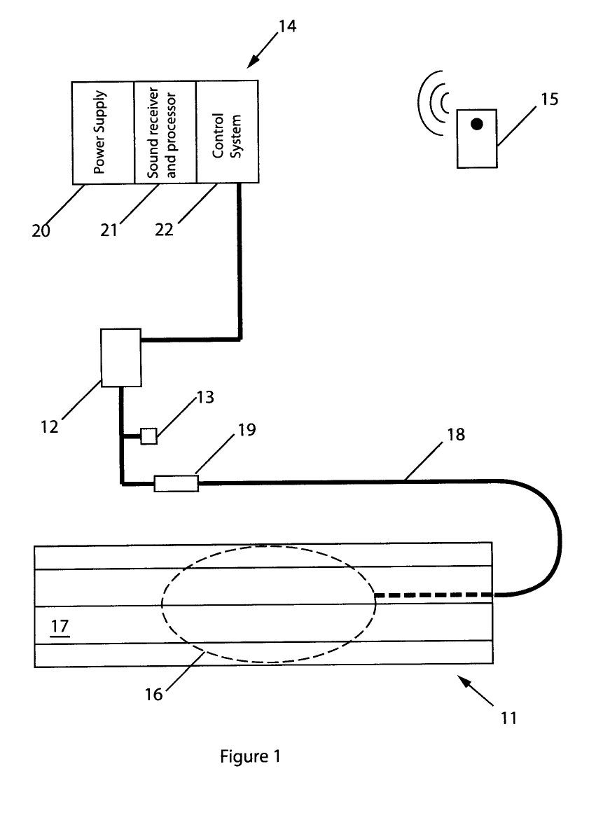

FIG. 1 illustrates a preferred embodiment of the present snore disrupting system, which comprises, an expansion unit 11, an air pressure supply or an inflator 12, at least one valve 13, an electronic circuit 14, and a wired or wireless audio-sensor and transmitter 15, which can be wired or wireless microphone transmitter or a cellphone or any other device suitable for audio sensing and analog or digital transmission. The expansion unit 11 comprises at least one inflatable bladder 16 positioned inside a sleeve assembly 17 comprised of hard surfaces. The expansion unit 11 could also be any other assembly of components that would allow for increase in volume when inflated and reduction of volume when deflated. The bladder 16 is further connected to the inflator 12 through at least one tube 18. The expansion unit 11 may be placed underneath a pillow, inside the pillowcase. The inflatable bladder 16 is inflated and deflated by the inflator 12 and through expanding and increasing in volume and deflating and reducing in volume changes the position of snorer's head, hence disrupting the snoring.

The pressure supply or the inflator 12 of the snore disrupting system pressurizes the bladder 16 through at least one tube 18, which may be a flexible plastic tube. The design of the air inflator 12 may be diaphragm, piston cylinder, blower, or others known to the person skilled in the art. The inflator 12 may be covered by soundproof material to reduce the noise.

The pressure supply or the inflator 12 is connected to at least one valve 13, which connects the outlet of inflator 12 to the atmosphere to depressurize the inflated bladder 16 while the inflator 12 is turned off. The valve 13 may be a normally open mini solenoid valve or other suitable valves known to the person skilled in the art. The pressure supply or the inflator 12 may further be connected to at least one silencer 19, which is connected to the outlet of the inflator 12 to reduce the transfer of the noise and vibration to the expansion unit 11.

The electronic circuit unit 14 of the snore disrupting system comprises a power supply 20, which could be a battery or an AC wall outlet or any other suitable sources of electrical power, a sound receiver and processor 21, and a control system 22. The electronic circuit unit 14 turns the inflator 12 on and off and controls the function of the snore disrupting system. The electronic circuit unit 14 may further comprise an adjustable on and off timer relay, as part of its control system, for setting the period of running the inflator 12. The electronic circuit unit 14 can be in part or whole a cellphone or any other device known to the person skilled in the art that can allow for sound processing and transmitting.

The sound receiver and processor 21 of the electronic circuit unit 14, receives what might be the snore sound from the audio-sensor and transmitter 15. The control system 22 of the electronic circuit unit 14 then turns on the inflator 12. The sound receiver and processor 21 may comprise a noise filter to eliminate the noise and may further comprise a voice recognizing memory or a voice detector to distinguishing the individual snorer's voice. The voice recognizing memory may be utilized when more than one person snores in the same room and separate snore disrupting system are used in close proximity. In this case, the system should recognize the individual user's snoring sound. The snore disrupting system may work with or without voice recognizing memory.

The snore sound is transmitted by wired or wireless audio-sensor and transmitter 15 or a cellphone or any other device that would allow for audio transmission, to the electronic circuit unit 14 and switches on the inflator 12 and closes the valve 13, resulting in the inflation of the expander unit's bladder 16.

What might be the snoring sound is detected by a wired or wireless audio-sensor and transmitter 15 or a cellphone or any other device that would allow for audio transmission, and is transmitted to the sound receiver and processor unit 21 that is in communication with the control system 22. When signaled by the sound receiver and processor unit 21, the control system 22 turns on the inflator 12 and closes the valve 13 for a predefined period of time, and then turns off the inflator 12 and opens the valve 13 for a predefined period of time. The total of the on and off time periods of the inflator 12 make one time cycle of the system. The running on and off of the inflator 12 may be done by a timer relay as part of the control system 22, receiving electrical signals from the sound receiver and processor unit 21.

By turning on the inflator 12, the bladder 16 is inflated, expanding the expander unit 11, causing the snorer's head that is located on the pillow to gently move up or in any other direction depending on the position of the head during a predefined period of time. After the predefined period of the inflator 12 being on and the expander unit 11 reaching a predefined maximum expansion, the inflator 12 is turned off by the control system 22, allowing the bladder 16 to depressurize through the valve 13, causing the expander unit 11 to reduce in cross sectional area, resulting in the snorer's head to move down or in any other direction depending on the position od the head.

In an alternative embodiment of the invention, the system does not have or make use of the audio-sensor and transmitter 15 and the sound receiver and processor 21 as part of the electronic circuit unit 14. In this embodiment the electronic circuit unit 14 may be set to periodically turn the inflator 12 on and off, independent of the snoring sound. The electronic circuit unit 14 may comprise an adjustable on and off timer relay for setting the period of running the inflator 12. By setting the timer for the period less than the gaps between when the devise disrupts the snoring and when the snoring starts again, the snorer's head is periodically moved.

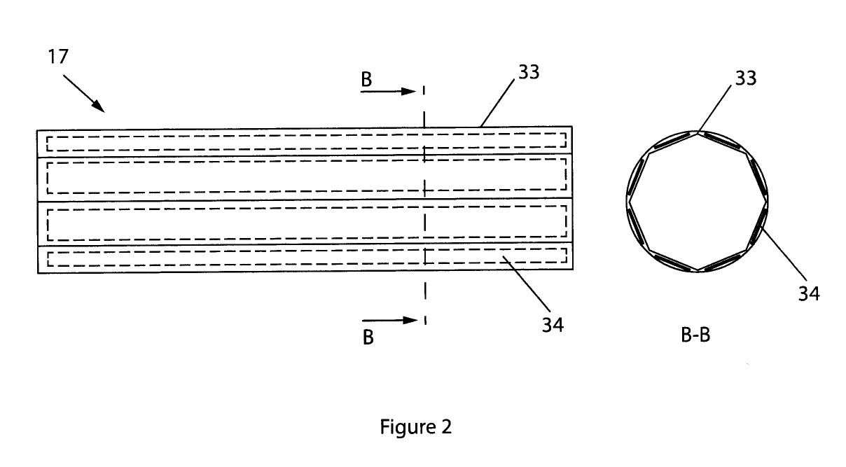

FIG. 2 illustrates a preferred embodiment of the sleeve assembly 17 of the expansion unit 11 of FIG. 1. The sleeve assembly 17 comprises a flexible sleeve structure 33 and a plurality of flat bars 34 or any other semi-rigid longitudinal structure. The flexible sleeve structure 33 may be made of flexible plastic or robust fabric with parallel longitudinal sacs. The flat bars 34 may be made of light and hard material and may be separated in two or many segments. By inserting one flat bar in each sac of the flexible sleeve structure 33, the sleeve assembly 17 may form into a reinforced body. By inflating the bladder 16 of FIG. 1 that would be placed inside the sleeve assembly 17, the sleeve assembly 17 may form a rigid cylindrical or polygonal shape sleeve, extending and longitudinally transferring the expansion motion of the inflatable bladder 16 of FIG. 1, pushing up the pillow to change the position of the snorer's head.

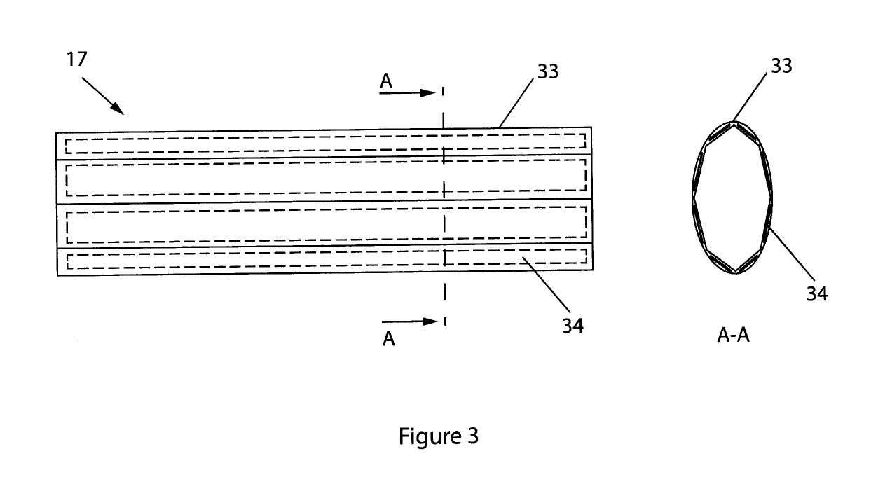

FIG. 3 illustrates the sleeve assembly 17 in a position when the bladder 16 of FIG. 1 has been deflated, resulting the sleeve assembly 17 to fold and reduce in cross sectional area, resulting in the snorer's head to move down and back to the initial position.

The above-described embodiments of the invention are intended to be examples of the present invention and alternations and modifications may be effected thereto by those of skill in the art, without departing from the scope of the present invention.

1. A snore disrupting system comprising:
an audio-sensor and transmitter as a means for capturing and transmitting the sound of snore;
an electronic circuit comprising an AC or DC power supply, a sound receiver and processor as a means for receiving and processing sound, and a control system as a means for controlling the function of the snore disrupting system, in communication with the said audio-sensor and transmitter;
an inflator as a means for supplying air pressure, and in communication with the said electronic circuit;
an expansion unit comprising at least one inflatable bladder and at least one sleeve assembly, in communication with the inflator through at least one tube, which may be directly or indirectly placed underneath the head of the person who's snoring is to be interrupted; and
a valve, which connect the outlet of the said inflator to the atmosphere as a means for deflating the said bladder;
wherein the said sleeve assembly comprises a flexible sleeve structure and a plurality of flat bars or any other semi-rigid longitudinal structure, the flexible sleeve structure being made of flexible plastic or robust fabric with parallel longitudinal sacs.

2. The snore disrupting system of claim 1, wherein by inflating the said bladder of the expansion unit, the sleeve assembly may form a rigid cylindrical or polygonal form, extending and longitudinally transferring the expansion motion of the inflatable bladder, pushing up the and changing the position of the snorers head

3. The snore disrupting system of claim 1, wherein the said expansion unit comprises at least one inflatable bladder positioned inside a sleeve assembly.

4. (canceled)

5. (canceled)

6. The snore disrupting system of claim 3, wherein the said flat bars which may be made of light and hard material and may be divided in to two or many segments are inserter in the said sacs, and together form a reinforced body

7. The snore disrupting system of claim 1, wherein the said sleeve assembly may be any semi-rigid structure that would allow for expansion in one or multiple directions

8. The snore disrupting system of claim 1, wherein the said expansion unit can be places in under a pillow and in a pillowcase, places under the head of the user

9. The snore disrupting system of claim 1, wherein the said audio-sensor and transmitter is in communication with the electronic circuit

10. The snore disrupting system of claim 1, wherein the said sound receiver of the electronic circuit receives sound from the audio-sensor and transmitter

11. The snore disrupting system of claim 1, wherein the said sound processor of the electronic circuit processes the sound received by the sound receiver to detect the snore sound

12. The snore disrupting system of claim 1, wherein the said sound processor signals the control system when snore sound is detected

13. The snore disrupting system of claim 1, wherein the said control system turns on the inflator and closes the valve of the for a set period of time

14. The snore disrupting system of claim 1, wherein the said inflator inflates the bladder of the expansion unit to a predefined set maximum expansion

15. The snore disrupting system of claim 1, wherein the said expansion unit expands and changes the position of the head of the user of the system, when triggered by the sound of snoring

16. The snore disrupting system of claim 1, wherein the said control system turns off the inflator and opens the said valve after full inflation, allowing the bladder to deflate for a set period of time, returning the expander unit to the initial position

17. A snore disrupting system comprising:
an electronic circuit comprising an AC or DC power supply, an adjustable on and off timer relay, and a control system as a means for controlling the function of the snore disrupting system an inflator as a means for supplying air pressure, and in communication with the said electronic circuit
an expansion unit comprising at least one inflatable bladder and at least one sleeve assembly, in communication with the inflator through at least one tube, which may be directly or indirectly placed underneath the head of the person who's snoring is to be interrupted
a valve, which connect the outlet of the said inflator to the atmosphere as a means for deflating the said bladder;
an expansion unit comprising at least one inflatable bladder and at least one sleeve assembly

18. The snore disrupting system of claim 15, wherein the said electronic circuit unit may be set to periodically turn the inflator on and off, independent of the snoring sound

19. The snore disrupting system in claim 15, wherein the said electronic circuit unit sets the period of running the inflator, where by setting the timer for the period less than the gaps between when the devise disrupts the snoring and when the snoring starts again, the snorer's head is periodically moved.
